(19) United States
(12) Patent Application Publication
(10) Pub. No.: US 2026/0250619 A1
(43) Pub. Date: Aug. 27, 2026

(54) CLOSED CHANNEL TYPE BIOPARTICLE PROCESSING SYSTEM AND CLOSED CHANNEL TYPE BIOPARTICLE PROCESSING METHOD

(71) Applicant: Sony Group Corporation, Tokyo (JP)

(72) Inventor: Tomoyuki Umetsu, Tokyo (JP)

(73) Assignee: Sony Group Corporation, Tokyo (JP)

(21) Appl. No.: 19/161,099

(22) PCT Filed: Feb. 21, 2024
(86) PCT No.: PCT/JP2024/006208

§ 371 Date: Aug. 29, 2025

(30) Foreign Application Priority Data
Mar. 8, 2023 (JP) 2023-035772

(51) Int. Cl. C12M 1/34; C12M 1/06; C12M 1/12

(52) U.S. Cl. CPC C12M 41/30; C12M 25/10; C12M 27/02

(57) ABSTRACT

Provided is a technology for executing a separation process and a coloring process for bioparticles in a closed space.

The present technology provides a closed channel type bioparticle processing system including stirring means that stirs a sample, separating means that uses magnetic beads each having a particle bonding substance, and concentration measuring means that measures a concentration of bioparticles in the sample. The stirring means and the separating means are automatically switchable. The bioparticles are colored after separation of the bioparticles by the separating means. The present technology also provides a closed channel type bioparticle processing method including a stirring step that stirs a sample, a separating step that uses magnetic beads each having a particle bonding substance, and a concentration measuring step that measures a concentration of bioparticles in the sample. The stirring step and the separating step are automatically switchable. The bioparticles are colored after separation of the bioparticles in the separating step.

### TECHNICAL FIELD

The present technology relates to a closed channel type bioparticle processing system and a closed channel type bioparticle processing method. The present technology particularly relates to a closed channel type bioparticle processing system and a closed channel type bioparticle processing method available for processing of a sample containing bioparticles.

### BACKGROUND ART

For example, modified beads which have antibodies or the like specifically bondable to a surface marker of bioparticles are used to condense bioparticles corresponding to a measurement target from a sample.

Generally, a method for separating bioparticles by using modified beads having antibodies or the like is classified into positive selection which captures target particles by using antibodies or the like corresponding to one surface marker of the target particles and negative selection which removes bioparticles other than target particles by using antibodies or the like corresponding to a surface marker of the bioparticles other than the target particles. According to these types of selection, separation between modified beads having antibodies or the like and bioparticles is conventionally achieved by a method utilizing magnetism or a filter (sieve).

For meeting a demand for finer selection of bioparticles, bioparticles are colored by using antibodies or the like which have fluorescent labels corresponding to a surface marker of the bioparticles, and separated or analyzed by flowcytometry. Separation and/or analysis of a specific group is achievable by the coloring of bioparticles. For example, T-cells can be colored by using CD3 antibodies having FITC labels. The colored T-cells can be separated and/or analyzed by using flowcytometry, for example. Flowcytometry analyzes all bioparticles one by one. Accordingly, extremely accurate purification and/or analysis is realizable.

Meanwhile, flowcytometry which individually supplies bioparticles into a microchannel and analyzes fluorescence amounts for separation generally requires a long time for separation of bioparticles corresponding to a measurement target in comparison with a method which collectively processes all bioparticles by using beads. Accordingly, a user is required to select a method having an appropriate application range from various methods including the foregoing two methods, and executes the selected method according to an application purpose in actual circumstances. Moreover, a step for selecting bioparticles is executed in a manufacturing process also in recent cell therapy and other fields, typically in cellular immune therapy. This step is performed for medical purposes.

Accordingly, it is preferable that processes be automatically executed within a closed space to eliminate mixture of foreign substances and uncertain factors caused by manual work.

For example, note herein that PTL 1 discloses a closed type membrane separation system which separates desired blood components from all blood by using a hollow fiber filter to collect and process the desired blood components.

### CITATION LIST

### Patent Literature

[PTL 1]

U.S. Pat. No. 10,918,780

### SUMMARY

### Technical Problem

As described above, it is preferable that processes such as separation and coloring of bioparticles corresponding to a measurement target be automatically performed within a closed space to eliminate mixture of foreign substances and uncertain factors caused by manual work. However, an automatic processing technology within a closed space is currently under development.

Accordingly, a main object of the present technology is to provide a technology capable of executing a separation process and a coloring process of bioparticles within a closed space.

### Solution to Problem

Initially, the present technology provides a closed channel type bioparticle processing system including stirring means that stirs a sample, separating means that uses magnetic beads each having a particle bonding substance, and concentration measuring means that measures a concentration of bioparticles in the sample. The stirring means and the separating means are automatically switchable. The bioparticles are colored after separation of the bioparticles by the separating means.

In addition, the present technology further provides a closed channel type bioparticle processing method including a stirring step that stirs a sample, a separating step that uses magnetic beads each having a particle bonding substance, and a concentration measuring step that measures a concentration of bioparticles in the sample. The stirring step and the separating step are automatically switchable. The bioparticles are colored after separation of the bioparticles in the separating step.

### BRIEF DESCRIPTION OF DRAWINGS

FIG. 1 is a schematic conceptual diagram illustrating a closed channel type bioparticle processing system arranged in a work flow order.

FIG. 2 is a schematic diagram illustrating a configuration of a closed channel type bioparticle processing device according one embodiment.

FIG. 3 is a conceptual diagram illustrating an outline of a measurement principle of concentration measuring means.

FIG. 4 is a schematic diagram illustrating an example of arrangement of stirring means, separating means, and a reservoir.

FIG. 5 is a diagram illustrating a flow example of a closed channel type bioparticle processing method using the closed channel type bioparticle processing system.

FIG. 6 is a conceptual diagram illustrating an example of an operation during negative selection of bioparticles from a sample processed by magnetic beads.

FIG. 7 is a conceptual diagram illustrating an example of an operation during positive selection using bioparticles bonded to magnetic beads.

FIG. 8 is a conceptual diagram illustrating an example of an operation during condensation of a sample with use of a hollow fiber membrane module.

FIG. 9 is a conceptual diagram illustrating an example of an operation during cleaning with use of the hollow fiber membrane module.

FIG. 10 is a schematic diagram illustrating respective modifications of the configuration of the closed channel type bioparticle processing device.

FIG. 11 is a diagram illustrating an example of a control flow for executing a concentration adjustment flow in a stable manner.

FIG. 12 is a schematic conceptual diagram illustrating a liquid supply mechanism of a tube pump.

FIG. 13 is a schematic conceptual diagram illustrating a method for feedback control of a liquid amount injected into the tube pump according to measurement values of the concentration measuring means.

FIG. 14 is a schematic diagram illustrating a modification of the configuration of the closed channel type bioparticle processing device.

FIG. 15 is a diagram illustrating an example of an operation during concentration adjustment performed on an assumption that the hollow fiber membrane module is used.

FIG. 16 is a photograph substituting for a drawing and illustrating an example of data of noise generated by dirt or bubbles in the closed channel during measurement by the concentration measuring means.

FIG. 17 is a photograph substituting for a drawing and illustrating an example of a liquid amount change produced when a cleaning process is executed for a sample containing bioparticles while setting the same number of rotations for the tube pump.

### DESCRIPTION OF EMBODIMENTS

Preferred embodiments for carrying out the present technology will be hereinafter described.

Note that the embodiments discussed hereinbelow are presented as typical embodiments of the present technology. It is therefore not intended that the scope of the present technology should be limited to only these embodiments.

Note that the description of the present technology will be presented in the following order.
1. First embodiment (closed channel type bioparticle processing system 1)
(1) Description of first embodiment
(2) Configuration example of closed channel type bioparticle processing device 2
(3) Configuration example of concentration measuring means
(4) Configuration example of stirring means and separating means
(5) Configuration example of hollow fiber membrane module 209 and cleaning means
(6) Description of particle bonding substance
(7) Flow example of closed channel type bioparticle processing method using system 1 of present technology
(8) Modifications of closed channel type bioparticle processing device 2
2. Automatic process using concentration measuring means
(1) Stabilization of concentration adjustment flow
(2) Stabilization of cleaning process flow
(3) Automatic process effect check
3. Second embodiment (closed channel type bioparticle processing method)

### 1. First Embodiment (Closed Channel Type Bioparticle Processing System 1)

A closed channel type bioparticle processing system 1 according to the present technology (hereinafter also simply referred to as a “system 1 according to the present technology”) will be hereinafter described in detail.

### (1) Description of First Embodiment

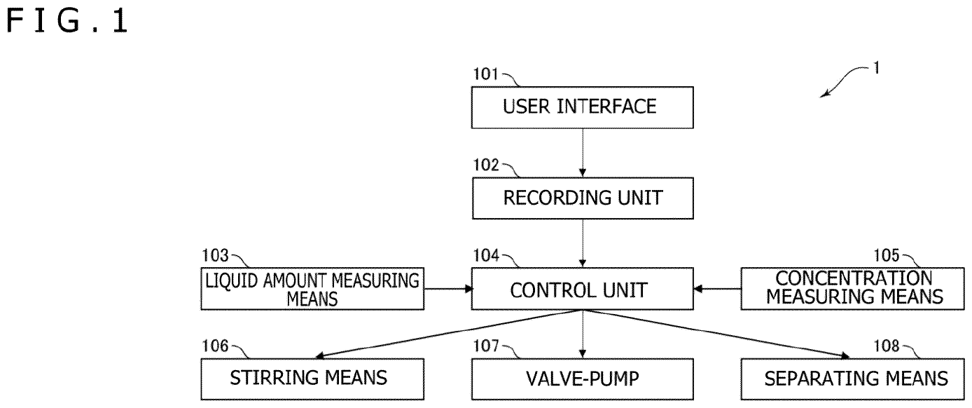

FIG. 1 is a schematic conceptual diagram illustrating the closed channel type bioparticle processing system 1 arranged in a work flow order. The system 1 according to the present technology includes stirring means 106 which stirs a sample, separating means 108 which uses magnetic beads each having a particle bonding substance, and concentration measuring means 105 which measures a concentration of bioparticles in the sample. In addition, the system 1 may further include a user interface 101, a recording unit 102, liquid amount measuring means 103, a control unit 104, a valve-pump 107, and others. The system 1 may further include cleaning means (not illustrated) and a display unit (not illustrated).

The workflow of the system 1 according to the present technology will be hereinafter discussed.

Initially, the user interface 101 receives a process instruction from a user, and the recording unit 102 records an automatic process procedure in response to reception of this instruction. The control unit 104 executes control of a closed channel type bioparticle processing device 2 described below.

Moreover, the control unit 104 receives input of information from the liquid amount measuring means 103 which measures a liquid amount of a tank constituting a channel, and from the concentration measuring means 105 which measures a concentration of bioparticles contained in a sample within the channel, to use the received information for determination at the time of execution of an automatic process. The control unit 104 transmits a control command chiefly to parts such as the valve-pump 107 for controlling liquid within the channel, the separating means 108 for achieving separation using magnetic beads, and the stirring means 106 for stirring bioparticles hardened in the sample. The above-described means is operated in accordance with the automatic process procedure recorded in the recording unit 102, to automatically execute a separation process and a coloring process for the bioparticles. Moreover, the control unit 104 may transmit the control command to the cleaning means (not illustrated) as necessary.

The user interface 101 is a part operated by a user such as an operator. The user accesses respective parts of the system 1 according to the present technology via the user interface 101 to control the respective parts of the system 1 according to the present technology. For example, the user interface 101 enables the user to set items displayed on a display unit and conditions associated with the respective means. The user interface 101 may be connected to the closed channel type bioparticle processing device 2 described below via a network.

Note that the user interface 101 is not an essential component for the system 1 according to the present technology. An external operation device may be connected instead of using the user interface 101. For example, the user interface 101 may include a mouse, a keyboard, a button, a touch panel, a portable information terminal, or the like.

The recording unit 102 is a part for storing various kinds of data. For example, the recording unit 102 stores any related items such as an automatic processing procedure, information associated with samples and bioparticles, and recordings of control commands of the control unit 104. The recording unit 102 may be connected to the closed channel type bioparticle processing device 2 described below via a network. Alternatively, the recording unit 102 may be provided in a cloud. In this case, respective items of information recorded in the recording unit 102 in the cloud may be shared by respective users via a network.

Note that the recording unit 102 is not an essential component for the system 1 according to the present technology. Various kinds of data may be stored using an external storage device or the like.

The control unit 104 controls operations of respective constituent elements of the system 1 according to the present technology. The control unit 104 controls on-off of driving of the respective constituent elements, driving intensity, and the like, in accordance with a predetermined program. For example, information received from the liquid amount measuring means 103, the concentration measuring means 105, and the like may be taken into consideration for this control. The control unit 104 may be constituted by an information processing device (computer). For example, functions of the control unit 104 may be achieved by a general-purpose computer. Moreover, the control unit 104 may be connected to the closed channel type bioparticle processing device 2 described below via a network.

Note that the control unit 104 is not an essential component for the system 1 of the present technology. The operations of the respective constituent elements may be controlled using an external information processing device or the like.

Moreover, the system 1 according to the present technology may include a display unit (not illustrated) for displaying various types of information. For example, the display unit can display any items such as progress of the automatic processing procedure, information associated with samples and bioparticles, and details of control commands of the control unit 104. The display unit may be connected to the closed channel type bioparticle processing device 2 described below via a network.

Note that the display unit is not an essential component for the system 1 according to the present technology. An external display device or the like may be used for display. For example, the display unit may be a display, a printer, a portable information terminal, or the like.

In the present description, “bioparticles” may be biological particles. For example, bioparticles can refer to particles constituting creatures. Bioparticles can be microparticles.

For example, bioparticles may be cells. Cells can include animal cells (blood cells, etc.) and plant cells.

Particularly, cells can be blood cells or tissue cells. For example, these blood cells may be white blood cells (for example, peripheral blood mononuclear cells, etc.), red blood cells, or platelets. The blood cells particularly include white blood cells. For example, white blood cells may be monocytes (macrophages), lymphocytes, neutrophils, basophils, and eosinophils. For example, cells may be floating cells such as T-cells and B-cells. For example, the tissue cells noted above may be adherent cultured cells, adherent cells separated from tissues, or the like. Moreover, cells can include tumor cells. Cells may be either cultivated cells or non-cultivated cells. For example, bioparticles can include cell clusters such as spheroids and organoids.

Bioparticles may be non-cellular biological components such as extracellular vesicles, particularly may be exosomes or micro vesicles, for example.

Bioparticles may be microorganisms or viruses. Microorganisms can include bacteria such as colon bacilli, and fungi such as yeast fungi. For example, viruses may be DNA viruses or RNA viruses, or viruses including or not including envelopes.

Bioparticles can include biological polymers such as nucleic acids, proteins, and complexes of these. For example, these biological polymers may be polymers extracted from cells, or polymers contained in blood samples or other liquid samples.

In the present description, a “sample” may be a sample of liquid containing bioparticles. For example, the sample can be liquid obtained from a creature, particularly biological fluid. For example, biological fluid may be blood, lymphatic fluid, tissue liquid (for example, inter-tissue fluid, intercellular fluid, interstitial fluid, etc.), celomic fluid (for example, serosal fluid, pleural effusion, ascites, pericardial effusion, cerebrospinal fluid (spinal fluid), joint fluid (synovial fluid) ), or the like. Moreover, liquid containing bioparticles may be liquid obtained from these types of biological fluid.

According to one embodiment of the present technology, the sample may be a blood-derived sample, particularly may be a sample containing white blood cells. Moreover, the blood-derived sample may be a blood sample obtained by a red blood cell separation process. Note that this blood sample is not required to be a sample from which red blood cells are completely removed and may be a sample containing blood red cells. For example, the blood sample may be a sample containing red blood cells which are included in blood collected from a living body and are reduced by a separation process.

### (2) Configuration Example of Closed Channel Type Bioparticle Processing Device 2

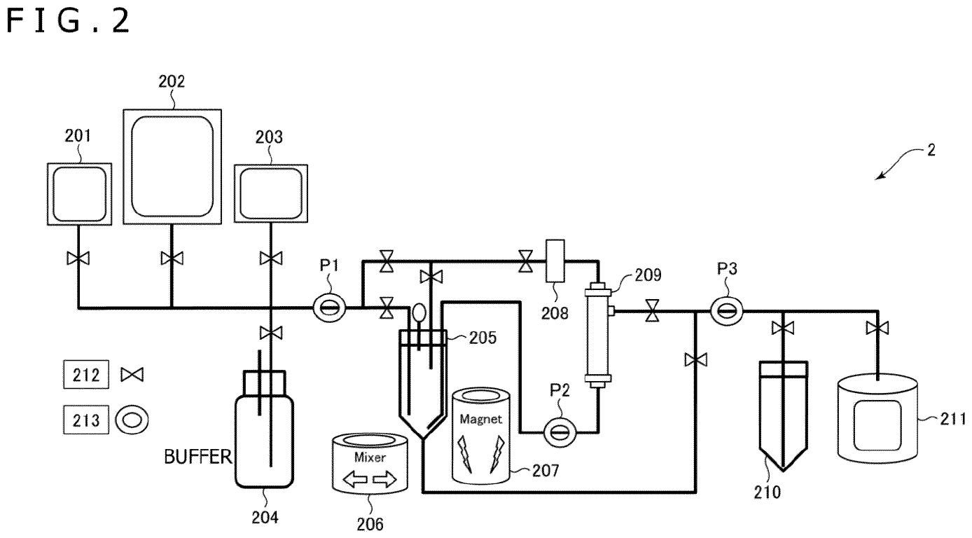

FIG. 2 is a schematic diagram illustrating a configuration of the closed channel type bioparticle processing device 2 according to one embodiment. The closed channel type bioparticle processing device 2 illustrated in FIG. 2 can automatically perform a separation process and a coloring process for bioparticles by using the system 1 according to the present technology. Note that the embodiment in FIG. 2 is one of embodiments using a magnetic bead reagent and a coloring reagent.

According to the embodiment in FIG. 2, a coloring reagent which contains bioparticle bonding substances having fluorescent labels and is formed by mixing a plurality of types of reagents according to the purpose of processing is filled into a container 201 (for example, bag, etc.) via a port. Similarly, a reagent which contains magnetic beads having bioparticle bonding substances and is formed by mixing a plurality of types of bead reagents according to the purpose of processing is filled into a container 203 (for example, bag, etc.) via a port. In addition, a sample containing bioparticles is set within a container 202 (for example, bag, etc.) via a port. The configuration according to the embodiment illustrated in FIG. 2 performs a sealing operation into these containers by using a sterile bonding device or the like. Accordingly, execution of all of the following processes can be completed within the closed channel type bioparticle processing device 2. Note that the sample containing the bioparticles may contain target particles corresponding to a separation target (also referred to as a “measurement target” in the present description) and non-target particles other than a separation target.

Buffers used at the time of suspension of the sample are filled into a container 204 (for example, bag, etc.) via a port. The buffers are used for cleaning of the sample, or for resuspension after a separation process or a condensation process. Reaction between the sample and the reagents is chiefly achieved within a reservoir 205 as a first reservoir. For example, the reservoir 205 has a cylindrical shape. In a case where sedimentation is caused within liquid during reaction with the reagents due to an excessively large size of the magnetic beads, stirring means 206 is attached to the reservoir 205 and operated to eliminate sedimentation.

For separating the bioparticles bonded to the magnetic beads, separating means 207 (for example, separation magnet 214, etc.) is brought close to the reservoir 205, making it possible to collect bioparticles having magnetic beads on a wall surface of the reservoir 205 and execute the separation process.

According to the embodiment illustrated in FIG. 2, a hollow fiber membrane module 209 is attached to the reservoir 205, making it possible to collect execute separation or condensation of the sample and a process for cleaning residues such as the remaining bead reagent. Moreover, concentration measuring means 208 which measures a concentration of bioparticles in the sample is attached between the hollow fiber membrane module 209 and the reservoir 205, making it possible to constantly measure the concentration of the bioparticles in the sample. Furthermore, filtrate discharged from the cleaning means is connected to a liquid waste container 211 (for example, bag, tank, etc.). The liquid waste container 211 is also connected to the reservoir 205 via a different channel. A reservoir 210 is disposed as a second reservoir between the reservoir 205 and the liquid waste container 211 to temporarily retain the processed bioparticles. As described above, one or a plurality of reservoirs may be disposed in the closed channel between the separating means 207 and the liquid waste container 211 in the present embodiment.

Moreover, the respective constituent elements constituting the system according to the embodiment in FIG. 2 are configured to automatically perform a series of cell processes by appropriately connecting a tube, a valve 212, and a pump 213 (preferably tube pump). Note that the respective numbers of the tube, the valve 212, and the pump 213 according to the present embodiment are not particularly limited to any number. One or a plurality of the tubes, the valves 212, and the pumps 213 may be included in the closed channel.

### (3) Configuration Example of Concentration Measuring

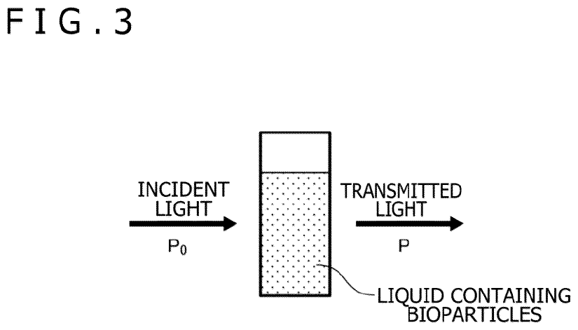

FIG. 3 is a conceptual diagram illustrating an outline of a measuring principle of the concentration measuring means 208. A transparent material may be adopted as a material of the tube constituting the channel to measure a change of transmitted light P from incident light Po, and a concentration of bioparticles contained in liquid within the tube can be measured. In other words, this configuration allows measurement of the concentration of the bioparticles through the tube. Accordingly, measurement is achievable even by processing within the closed channel.

### (4) Configuration Example of Stirring Means and Separating Means

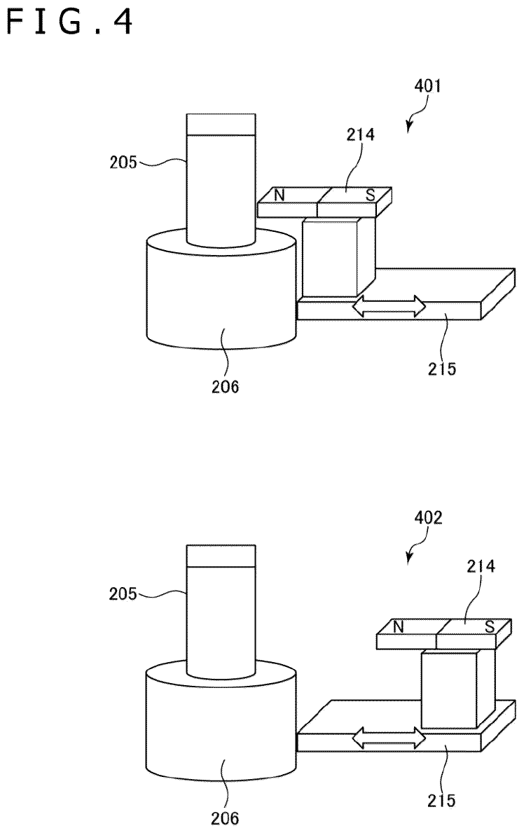

FIG. 4 is a schematic diagram illustrating an example of arrangement of the stirring means 206, the separating means 207, and the reservoir 205. The reservoir 205 is disposed on the stirring means 206 including a stirrer or the like. On-off of a stirring operation, intensity of stirring, and the like can be controlled in accordance with a control command given from the control unit 104 described in “(1) Description of first embodiment.” Moreover, the separating means 207 including a separation magnet 214 and the like is disposed in a state close to the reservoir 205 during a separation operation as indicated by 401 in FIG. 4, and is shifted to a state separated from the reservoir 205 during resuspension of the stirred and separated bioparticles as indicated by 402 in FIG. 4. Such a configuration can automatically achieve switching between the stirring means 206 and the separating means 207. Note that the separating means 207 further includes a stage 215 and the like for controlling a shift operation of the separation magnet 214 and the like, for example, and can operate the stage 215 and the like in accordance with a control command from the control unit 104.

### (5) Configuration Example of Hollow Fiber Membrane Module 209 and Cleaning Means

For example, the hollow fiber membrane module 209 includes an inlet, a container, an outlet, and a liquid waste outlet. Moreover, hollow fiber membranes are filled into the container.

The hollow fiber membranes contained in the hollow fiber membrane module 209 may be appropriately selected by a person skilled in the art. For example, the hollow fiber membranes may include mPES (modified polyethersulfone, ME (mixed cellulose ester), PES (polyether sulfone), or PS (polysulfone). For example, a hole size of the hollow fiber membranes expressed as MWCO (Molecular Weight Cut Off) may be in a range from 1 to 1,000 kD (inclusive), particularly from 2 to 900 kD (inclusive), more particularly from 3 to 800 kD (inclusive). For example, a hole size of the hollow fiber membranes may be in a range from 0.1 to 1.0 μm (inclusive), particularly from 0.15 to 0.9 μm (inclusive), more particularly from 0.2 to 0.8 μm (inclusive).

The hollow fiber membrane module 209 is configured to circulate the sample within the reservoir 205. For example, the hollow fiber membrane module 209 may be configured to be capable of controlling the bioparticle concentration of the sample within the reservoir 205, and may be configured to be capable of increasing or decreasing this bioparticle concentration.

For example, as presented in the embodiment in FIG. 2, the hollow fiber membrane module 209 is connected to the reservoir 205 via a circulation channel. Liquid within the reservoir 205 (particularly, sample) may be supplied to the hollow fiber membrane module 209 via the channel. Meanwhile, the sample having passed through the hollow fiber membrane module 209 may be supplied to the reservoir 205 via a different channel.

As described above, it is preferable in the present embodiment that a circulation channel is provided to circulate bioparticles between the reservoir 205 and the hollow fiber membrane module 209. Note that the circulation direction may be the opposite direction to the illustrated direction.

According to the present embodiment, it is preferable that the hollow fiber membrane module 209 described above be employed as the cleaning means. A cleaning method using the hollow fiber membrane module 209 will be detailed in S505 in the following “(7) Flow example of closed channel type bioparticle processing method using system 1 of present technology.”

### (6) Description of Particle Bonding Substance

The “particle bonding substances” in the present description may be substances used for capturing bioparticles. For example, the particle bonding substances may be substances bondable to bioparticles, or substances capturing bioparticles by using different substances. In the latter case, bioparticles may be bonded not to the substances capturing bioparticles but to the different substances.

As described above, the particle bonding substances are substances bondable to bioparticles. For example, the particle bonding substances may be antibodies or antibody fragments, particularly antibodies or antibody fragments bondable to antigens existing on surfaces of bioparticles, more particularly antibodies or antibody fragments bondable to surface antigens of cells.

As described above, the substances capturing bioparticles by using the different substances are not required to be substances bondable to bioparticles. For example, the foregoing substances may be substances bondable to particle bonding substances, particularly proteins bondable to antibodies or antibody fragments, more particularly proteins specifically bondable to antibodies or antibody fragments. For example, these proteins may be antibody bonding proteins or the like. For example, antibody bonding proteins may be any one or two or more types of proteins selected from protein A, protein G, protein L, and protein A/G.

Moreover, the particle bonding substances may be appropriately selected by a person skilled in the art according to the type of bioparticles corresponding to a separation target (particularly, substances existing on surfaces of bioparticles). Furthermore, the particle bonding substances can include labeled particle bonding substances having labels. For example, the particle bonding substances may be labeled antibodies or labeled antibody fragments. For example, the labels may be fluorescent labels such as fluorescent dye and fluorescent protein, enzyme labels such as HRP (Horseradish peroxidase) and AP (Alkaline phosphatase), labels including biotin, or labels including gold colloid. According to the present embodiment, particle bonding substances having fluorescent labels are more preferable in these examples. Note that the “magnetic beads having particle bonding substances” in the present description refer to beads constituted by magnetic beads selected as carriers, and particle bonding substances bonded to the magnetic beads.

### (7) Flow Example of Closed Channel Type Bioparticle Processing Method Using System 1 of Present Technology

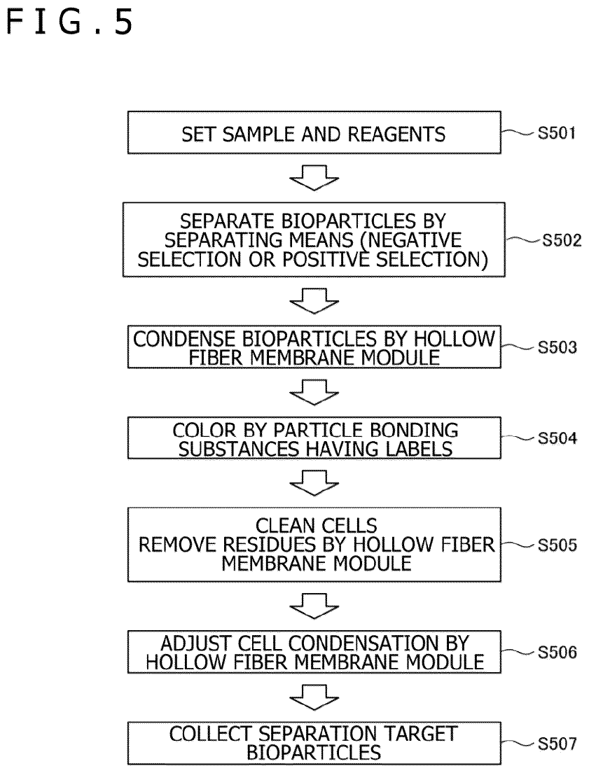

Described hereinafter with reference to FIG. 5 will be a flow example of the closed channel type bioparticle processing method using the system 1 according to the present technology. Performed in the present flow example are at least a process for separating bioparticles corresponding to a separation target from a sample containing these bioparticles and bioparticles other than the separation target, and a process for coloring the separated bioparticles by antibodies having fluorescent labels.

For example, the sample in the present flow example may be a blood-derived sample, particularly may be a sample containing white blood cells. Moreover, the sample particularly may be a blood-derived sample obtained by a red blood cell separation process. Note that the blood-derived sample is not required to be a sample from which red blood cells are completely removed, and may be a sample containing blood red cells. For example, the blood-derived sample may be a sample containing red blood cells which are included in blood collected from a living body and are reduced by a separation process.

As illustrated in FIG. 5, respective steps S501 to S507 are carried out in the present flow example.

Specifically, the present flow example is a flow example which automatically performs negative selection and fluorescent coloring with use of magnetic beads, for the sample containing the bioparticles corresponding to a separation target. Moreover, reference to the closed flow channel type bioparticle processing device 2 of the embodiment illustrated in FIG. 2 will be also made as necessary.

In S501, a sample containing bioparticles as a separation target and respective reagents are attached to the closed channel type bioparticle processing device 2. The reagents may be appropriately selected according to purposes of separation and coloring. In a case where distinction between the bioparticles by a plurality of markers is required, for example, it is preferable that a plurality of reagents be mixed before setting. Moreover, it is preferable that these operations be joined to each other in a sterile environment by using a sterile device, a laminar flow cabinet, or the like.

In S502, negative selection is executed using the set sample and the set reagent containing magnetic beads. Initially, this process mixes the sample and the reagent containing the magnetic beads by controlling the pump 213 and the valve 212 provided within the closed channel, and introduces the mixture into the reservoir 205. In this step, the liquid amount of the reagent containing the magnetic beads often decreases, in which state supply of the liquid becomes difficult. However, losses of the reagent containing the magnetic beads within the piping can be reduced by shifting the sample to a port of the reagent containing the magnetic beads, and shifting the sample and the reagent in a mixed state to the reservoir 205.

A certain reaction time is required in S502 to cause reaction between the magnetic beads and the sample within the reservoir 205. Accordingly, it is preferable that the liquid within the reservoir 205 be mixed at fixed intervals by using the stirring means 206 to prevent sedimentation of the bioparticles and promote bonding of the bioparticles.

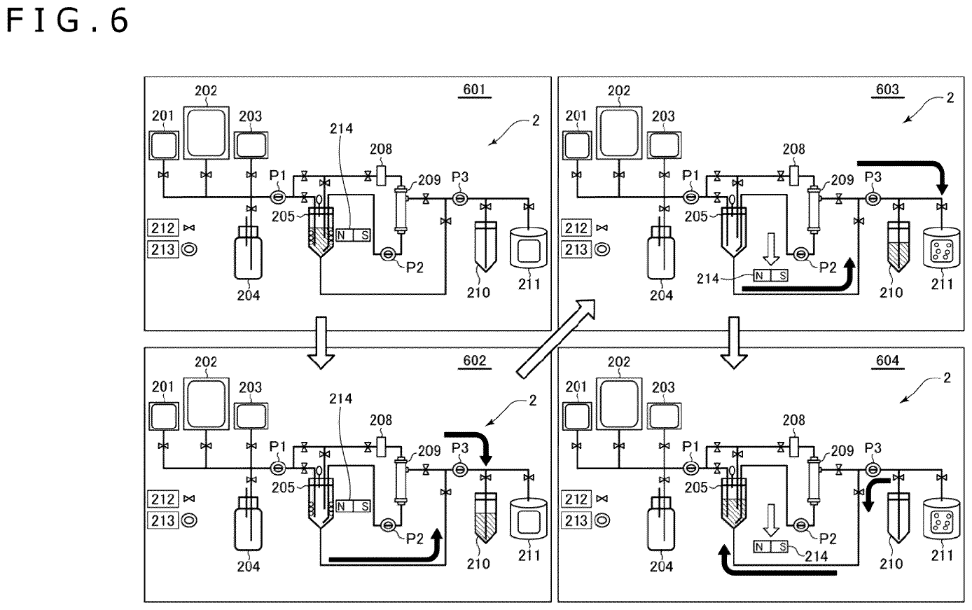

FIG. 6 is a conceptual diagram illustrating an example of an operation during negative selection of bioparticles from a sample processed by magnetic beads. As indicated by 601 in FIG. 6, bioparticles bonded to the magnetic beads are concentrated on the wall surface of the reservoir 205 by bringing the separation magnet 214 close to the reservoir 205. In this state, the bioparticles negatively separated within the reservoir 205 are shifted to the different reservoir 210 as indicated by 602 in FIG. 6. Thereafter, the separation magnet 214 is isolated from the reservoir 205, and buffers are introduced into the reservoir 205 and mixed using the stirring means 206 to suspend the bioparticles bonded to the magnetic beads.

Subsequently, liquid containing these magnetic beads is discharged to the liquid waste container 211 as indicated by 603 in FIG. 6. Finally, the bioparticles negatively separated and shifted to the different reservoir 210 are returned to the reservoir 205 from which the magnetic beads have been removed as indicated by 604 in FIG. 6. In this manner, negative selection of the sample with use of the magnetic beads is completed within the closed channel.

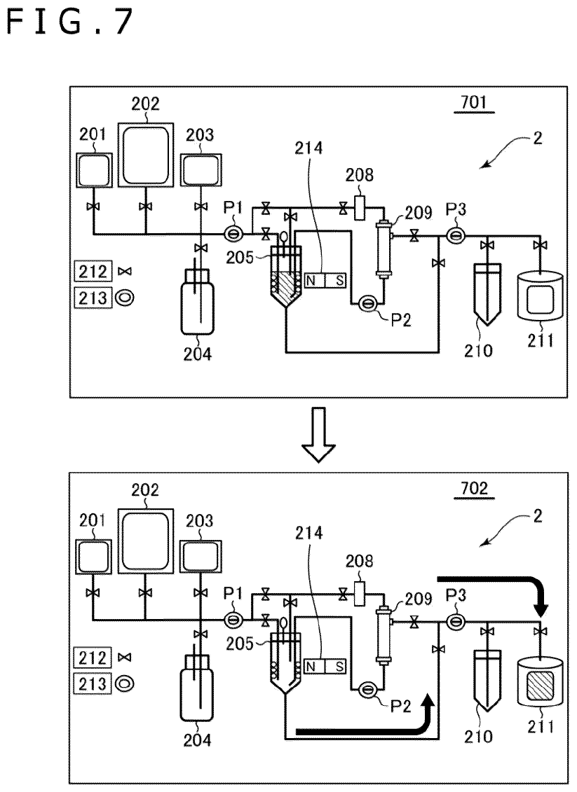

FIG. 7 is a conceptual diagram illustrating an example of an operation during positive selection using bioparticles bonded to magnetic beads. The process of positive selection is completed only by discharging liquid from the reservoir 205 to the liquid waste container 211 as indicated by 702 in FIG. 7 while bringing the separation magnet 214 close to the reservoir 205 as indicated by 701 in FIG. 7.

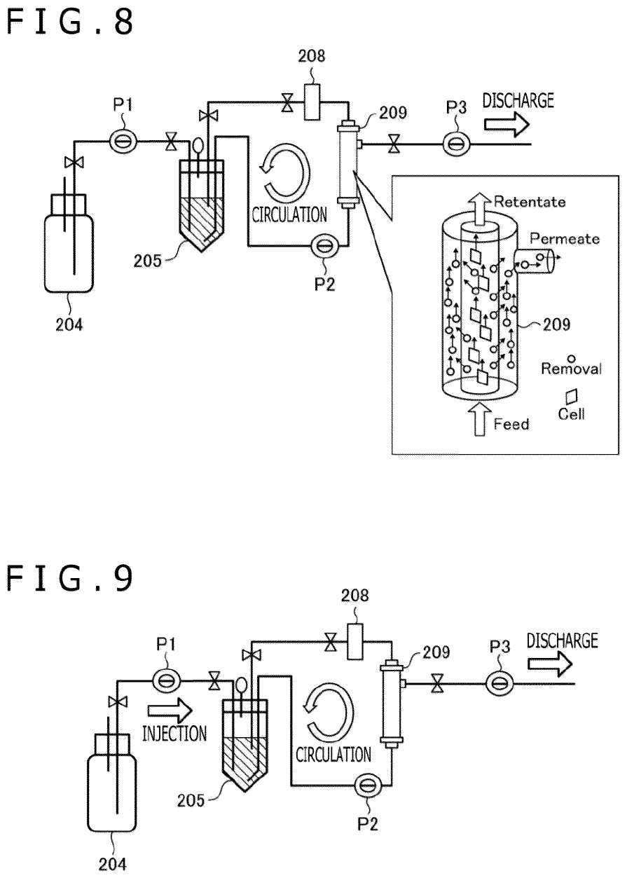

In S503, the sample obtained by processing with the magnetic beads is condensed by using the hollow fiber membrane module 209. This process is not an essential process, but is preferably carried out to reduce a reagent amount necessary for coloring in a subsequent step. FIG. 8 is a conceptual diagram illustrating an example of an operation during condensation of the sample with use of the hollow fiber membrane module 209. As illustrated in FIG. 8, the sample within the reservoir 205 is circulated in the hollow fiber membrane module 209 by using the circulation channel described above. In this state, buffers not containing the bioparticles are discharged from the discharge port of the hollow fiber membrane module 209 to increase the concentration of the bioparticles. It is preferable that the condensation of the bioparticles reaches a concentration range approximately from 1E7/mL to 1E8/mL, for example. This range is variable according to the types of the coloring reagents and the bioparticles.

In S504, the bioparticles are colored by labeled particle bonding substances (particularly, antibodies having fluorescent labels). Also in this step, there is a possibility that liquid supply becomes difficult due to a decrease in the liquid amount of the coloring reagent as in the reaction with the magnetic beads. Accordingly, the sample is shifted to the port of the coloring reagent and mixed with the coloring reagent before shifted to the reservoir 205. In this manner, losses of the coloring reagent within the piping decrease. Thereafter, the sample mixed with the coloring reagent comes into a bonding waiting state within the reservoir 205 for a certain period. The coloring carried out herein may be coloring for indicating the form of the bioparticles, or coloring for indicating substances included in the bioparticles (for example, surface antigens, etc.). For example, the coloring may be HE (Hematoxylin-Eosin) coloring, immunohistochemistry coloring, or the like.

In S505, cleaning is carried out by using cleaning means to remove unnecessary reagents from the sample containing the colored bioparticles. This process is not an essential process, but is preferably carried out to remove residues such as the remaining bead reagent and obtain a highly pure sample. FIG. 9 is a conceptual diagram illustrating an example of an operation during cleaning with use of the hollow fiber membrane module 209. As illustrated in FIG. 9, the cleaning is executed by discharging buffers while circulating the sample by using the hollow fiber membrane module 209, and simultaneously continuing addition of buffers. At this time, the cleaning is achievable more efficiently as the amount of liquid of the sample is smaller. Accordingly, the cleaning may be executed after condensation of the sample.

In S506, the concentration of the bioparticles corresponding to the separation target in the sample is adjusted to a desired concentration. The concentration adjustment is completed by monitoring measurement values of the concentration measuring means 208 during the foregoing condensation process described above and illustrated in FIG. 8, and stopping the condensation when the concentration reaches a target concentration. In addition, in a case where the target concentration is set to a low concentration, the measurement values of the concentration measuring means 208 are monitored while adding buffers, and dilution is ended when the condensation reaches a desired concentration. A series of processes for the separation process of the bioparticles corresponding to a separation target with use of the magnetic beads, and the coloring process of the separated bioparticles can be automatically executed within the closed channel by the foregoing series of operations without communicating with an external environment (for example, external air, external liquid, etc.). Accordingly, various advantageous effects such as reduction of human errors, cost reduction, time reduction, and sample contamination prevention can be achieved.

In S507, the sample containing the bioparticles as the separation target is collected. In this step, the reservoir 205 containing the collected sample may be extracted from the closed channel type bioparticle processing device 2 in a sterile environment by using a sterile device, a laminar flow cabinet, or the like, and used for sorting or analysis of the bioparticles as necessary. For example, the reservoir 205 may be used for a sample attached to a device performing sorting or analysis of bioparticles within a closed space, or may be used for a sample attached to a device performing sorting or analysis of bioparticles within an open space. The device performing sorting or analysis of bioparticles within a closed space may be a microparticle sorting device described in JP 2020-76736A, for example. However, the device according to the present embodiment is not limited to this example. The device performing sorting or analysis of bioparticles within an open space may be a microparticle measuring device described in JP 2020-51936A, for example. However, the device according to the present embodiment is not limited to this example. The system 1 according to the present technology may be used for preprocessing of a sample attached to the sorting or analyzing device noted above.

### (8) Modifications of Closed Channel Type Bioparticle Processing Device 2

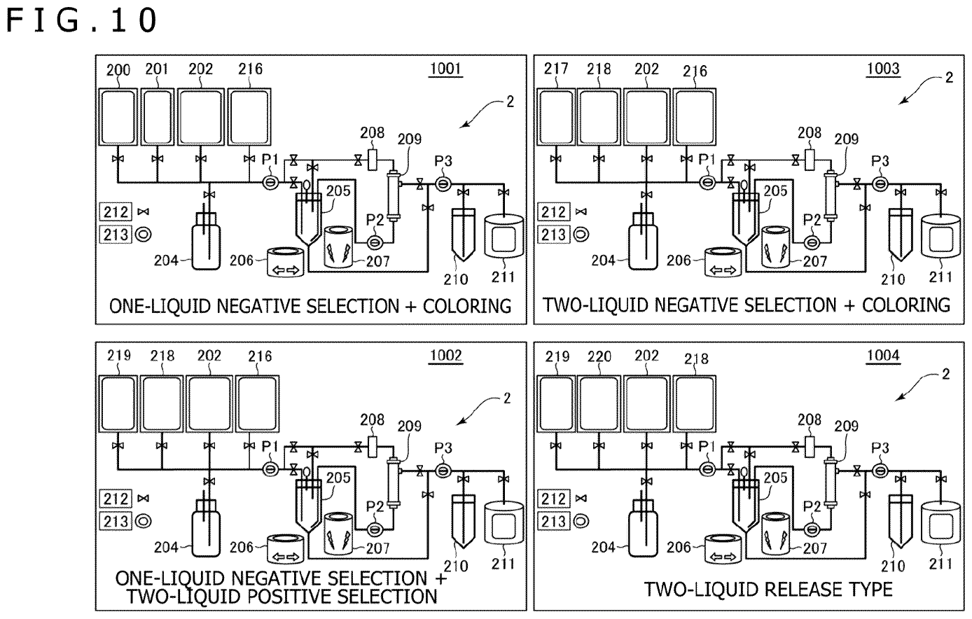

The automatic process described in “(7) Flow example of closed channel type bioparticle processing method using system 1 of present technology” is an operation example performed on an assumption that the reagent containing the magnetic beads is a reagent of one-liquid type. Note herein that many products contain two separated types of reagents for each, i.e., main bodies of magnetic beads, and antibody portions bonded to bioparticles, depending on types of reagents. FIG. 10 illustrates respective configuration examples of the closed channel type bioparticle processing device 2 used for automatically processing these reagents.

One port used by a user to set a reagent and a sample may be added to provide four ports in total and carry out many patterns of processing by using these ports. Indicated by 1001 in FIG. 10 is a case of one-liquid magnetic beads. In this case, one port corresponds to a not-used port. Specifically, the four containers including the four ports are constituted by a not-used container 200, the coloring reagent container 201, the sample container 202, and a reagent container 216 for a reagent containing magnetic beads for negative selection. A one-liquid negative selection process and a coloring process can be executed by using the closed channel type bioparticle processing device 2 having the configuration indicated by 1001.

Indicated by 1002 in FIG. 10 is a pattern for successively performing a process using magnetic beads, and corresponding to a one-liquid reagent for negative selection, and a two-liquid reagent for positive selection. Specifically, the four containers including the four ports are constituted by a particle bonding substance container 219 for positive selection, a reagent container 218 for a reagent containing magnetic beads for positive selection, the sample container 202, and the reagent container 216 for a reagent containing magnetic beads for negative selection. A one-liquid negative selection process and a two-liquid positive selection process can be executed by using the closed channel type bioparticle processing device 2 having the configuration indicated by 1002.

Indicated by 1003 in FIG. 10 is such a case where the magnetic beads are two-liquid beads. Specifically, the four containers including the four ports are constituted by a particle bonding substance container 217 for negative selection, the reagent container 218 for a reagent containing magnetic beads for positive selection, the sample container 202, and the reagent container 216 for a reagent containing magnetic beads for negative selection. A two-liquid negative selection process and a coloring process can be executed by using the closed channel type bioparticle processing device 2 having the configuration indicated by 1003.

Indicated by 1004 in FIG. 10 is such a case where a release reagent for releasing the magnetic beads is provided. Specifically, the four containers including the four ports are constituted by the particle bonding substance container 219 for positive selection, a release reagent container 220, the sample container 202, and the reagent container 218 for a reagent containing magnetic beads for positive selection. A two-liquid release process can be executed by using the closed channel type bioparticle processing device 2 having the configuration indicated by 1004.

As apparent from the above cases, input to three or a desired number larger than three of ports allows use of partially the same or completely the same channel configuration. In this case, various processes can be practiced with use of hardware having the same or only a partially modified pattern of tubes constituting the closed channel by appropriately changing reagents and the like set for the respective ports and also appropriately changing sequences of the automatic process. Moreover, achievable is such an advantageous effect that only replacement of a container part is required even for incorrect use of a magnetic bead reagent or the like.

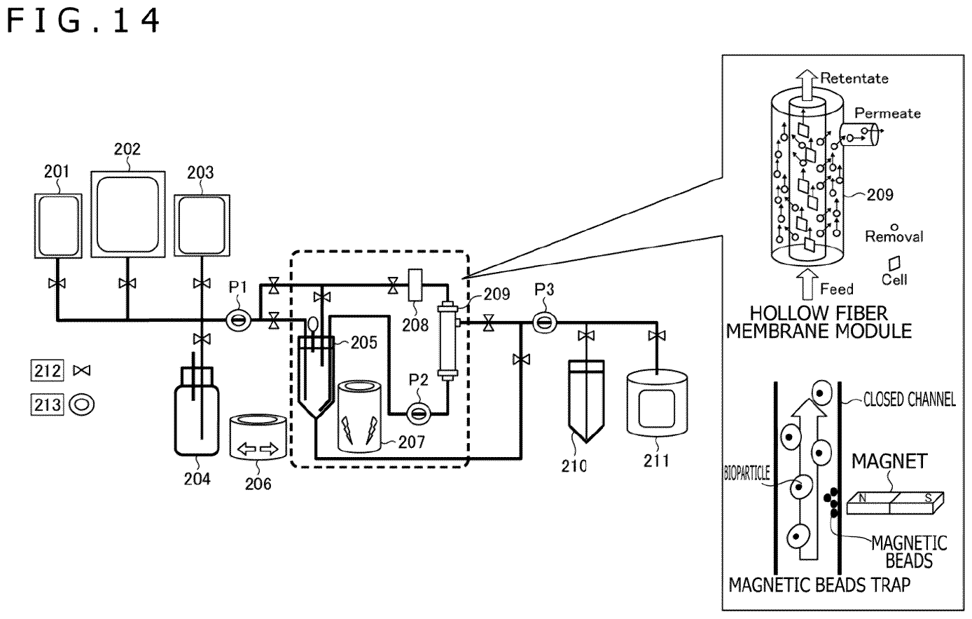

Furthermore, according to the present embodiment, the condensation process and the cleaning process are assumed to be performed by the hollow fiber membrane module 209 after separation using magnetic beads. Accordingly, there is a possibility that minute magnetic beads remain in principle. These remaining magnetic beads may cause adverse effects such as clogging of the hollow fiber membranes, depending on the hole size of the hollow fiber membrane module 209 and the size of the magnetic beads. FIG. 14 is a schematic diagram illustrating a modified example of the configuration of the closed channel type bioparticle processing device 2. As illustrated in FIG. 14, magnetic beads remaining on the magnet are attracted by bringing the magnet close to the channel located before entrance into the hollow fiber membrane module 209. In this manner, the remaining magnetic beads can be trapped before entrance into the hollow fiber membrane module 209. Accordingly, the adverse effects described above are avoidable.

### 2. Automatic Process Using Concentration Measuring Means

Hereinafter described in detail will be the automatic process using the concentration measuring means which is one of the constituent elements of the system 1 according to the present technology.

### (1) Stabilization of Concentration Adjustment Flow

One of important functions of the system 1 according to the present technology is concentration adjustment applied to bioparticles contained in a collected sample, and achieved in a closed space by using the concentration measuring means. This function is important because a bioparticle concentration considerably influences a processing time and performance during cell sorting executed within the closed channel for a sample preprocessed using the system 1 according to the present technology.

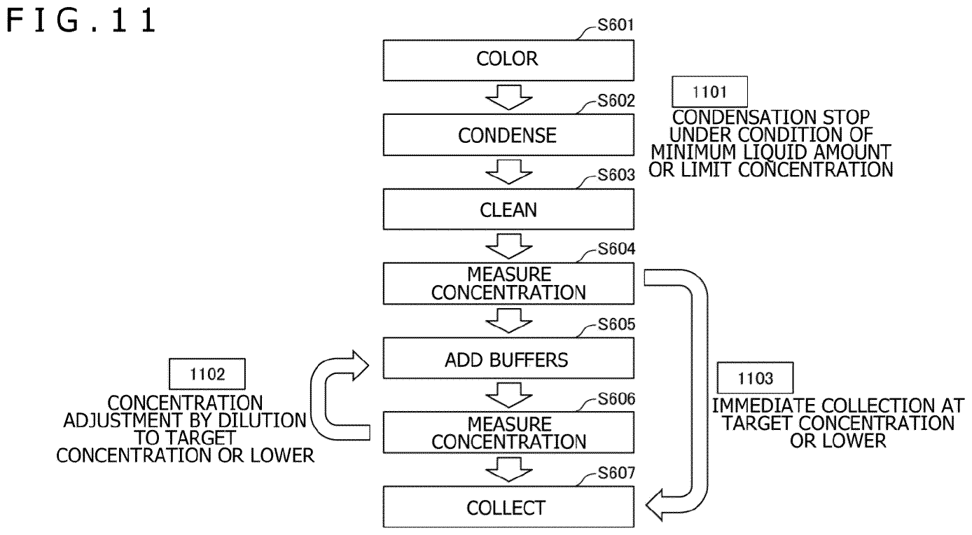

FIG. 11 is a diagram illustrating an example of a control flow for executing a concentration adjustment flow in a stable manner. Specifically, FIG. 11 illustrates the processing flow in and after S504 in FIG. 5 in more detail. After the coloring process is executed and completed in S601, the hollow fiber membrane module 209 in S602 condenses the sample containing the bioparticles to a closest possible concentration to a limit concentration as preprocessing so as to increase cleaning efficiency. An example method for setting this target concentration is indicated by 1101 in FIG. 11.

In the example of 1101 in FIG. 11, a minimum liquid amount (e.g., 10 mL, preferably 5 mL) or a limit concentration (e.g., 1E9 mL, preferably 1E8/mL) is designated as a setting condition. The condensation process is stopped when the liquid amount becomes smaller than the minimum liquid amount, or when the concentration exceeds the limit concentration. Note that these setting conditions are appropriately set while considering a minimum sample liquid amount which is allowed to be handled by a device performing sorting or analysis of bioparticles and is variable according to a length of piping or the like of the device, and a concentration limit in excess of which viscosity and a survival rate are influenced depending on types of bioparticles. According to the present embodiment, therefore, an arrival at either of these limits is designated as a criterion for stopping condensation immediately after coloring.

Thereafter, cleaning by buffer exchange is performed in S603, and then the concentration is adjusted to a final bioparticle concentration designated by the user by using the concentration measuring means 208 in S604. In this step, in a case where the concentration of the bioparticles is higher than the target concentration designated by the user, processing in S605 and S606 using a setting method indicated by 1102 in FIG. 11 by way of example is carried out. Specifically, buffers are added to dilute the sample in S605, and the concentration of the bioparticles is again measured in S606. The processing in S605 and S606 is continued until the concentration becomes the target concentration or lower. After the concentration becomes the target concentration or lower, the process shifts to a collection process as indicated by S607. Note that adopted herein is the method for reducing the concentration to the target concentration or lower by dilution because a probability of an error of the concentration measuring means 208 caused by dust, bubbles, or the like is higher in a case of use of condensation for reduction of the concentration to the target concentration than in a case of use of dilution. Accordingly, the foregoing process is adopted to avoid such an error.

Meanwhile, in a case where the concentration of the bioparticles is lower than the target concentration designated by the user, a setting method indicated by 1103 in FIG. 11 by way of example is adopted. Specifically, the process shifts to the collection process in S607 while skipping the processing in S605 and S606.

According to the control using the foregoing setting methods indicated by 1102 and 1103 in FIG. 11, condensation to the limit condition is achieved before concentration adjustment, and only the dilution process is performed as a subsequent process instead of either one of two choices of concentration and dilution. Therefore, the channel operations are to be one operation. Moreover, this concentration adjustment method can avoid a risk of a liquid decrease to an amount smaller than a minimum liquid amount allowed to be handed by the device performing sorting or analysis of the bioparticles. Furthermore, this concentration adjustment method offers an advantageous effect of eliminating a necessity of using a difficult method for measuring a liquid amount on the basis of a height of a liquid surface, and also an advantageous effect of reducing influences of bubbles.

### (2) Stabilization of Cleaning Process Flow

Described in “(1) Stabilization of concentration adjustment flow” have been the advantageous effects achievable by the method for condensing bioparticles to a limit before the cleaning process. However, for executing the cleaning process after reduction of a liquid amount to a limit allowed to be handled by the device performing sorting or analysis of bioparticles, highly accurate control of both a liquid amount supplied from the pump 213 and a discharged liquid amount is required. Specifically, equalization is required between a buffer liquid amount injected from a pump Pl and a liquid amount discharged from a pump P3 in the cleaning process illustrated in FIG. 9.

Note herein that the respective processes performed by the system 1 according to the present technology are assumed to be carried out within the closed channel.

Accordingly, the pump 213 needs to be used for liquid supply to prevent direct contact between liquid and the device and the like. For example, this pump may be a tube pump, preferably a peristaltic pump.

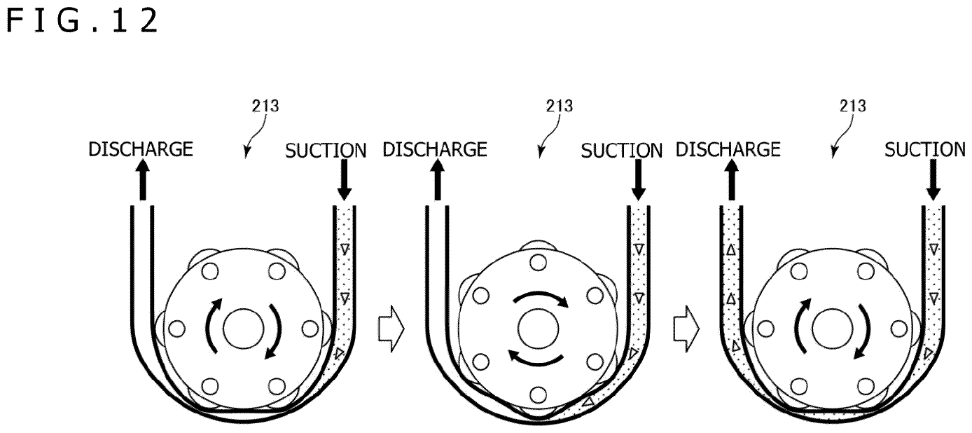

FIG. 12 is a schematic conceptual diagram illustrating a liquid supply mechanism of the tube pump. The tube pump uses a method adopting such a mechanism that liquid inside the tube is supplied by crushing the tube. In this case, the liquid supply amount is controlled according to the number of rotations of the pump. However, accurate control of the liquid supply amount is difficult due to variations of the inside diameter or the shape of the attached tube.

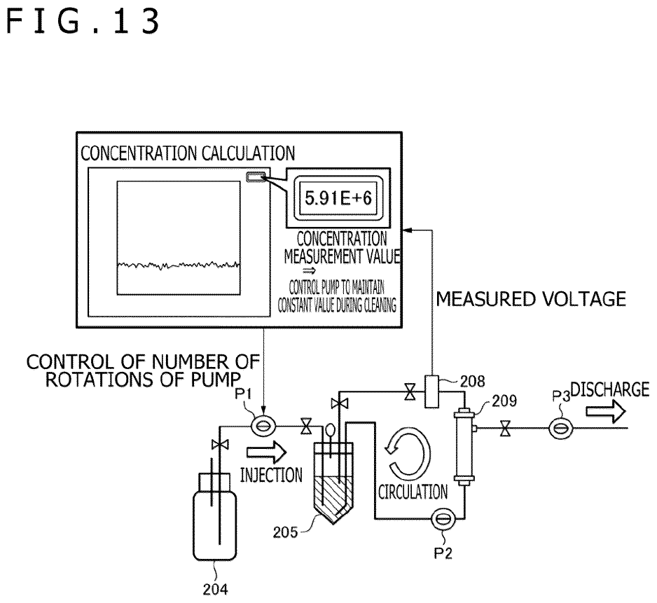

FIG. 13 is a schematic conceptual diagram illustrating a method for feedback control of a liquid amount injected into the tube pump according to measurement values of the concentration measuring means 208. The control method illustrated in FIG. 13 is an effective control method for solving the above problem. The system 1 according to the present technology is configured to constantly circulate bioparticles during the cleaning operation. Accordingly, a concentration of bioparticles is measurable in real time by the concentration measuring means 208. Specifically, the concentration measuring means 208 is capable of controlling the concentration of the bioparticles within the closed channel such that the concentration is constantly maintained at a fixed concentration. Losses of the bioparticles within the sample are not produced by the hollow fiber membrane module 209. Accordingly, a state where the concentration of the bioparticles is maintained at a constant concentration is equivalent to a state where the injection liquid amount is maintained at a constant amount.

The control herein is such control that the injection liquid amount of the pump P1 is raised (i.e., the number of rotations of the pump P1 is raised) in a case of an increase in the concentration of the bioparticles, and that the injection liquid amount of the pump P1 is reduced (i.e., the number of rotations of the pump P1 is reduced) in a case of a decrease in the concentration of the bioparticles.

In this manner, cleaning can be stably executed with use of a small amount of liquid under the control utilizing measurement values of the concentration measuring means 208 at the time of the sample cleaning process using the hollow fiber membrane module 209. Moreover, a desired cleaning process can be stably executed without a necessity of additionally attaching a liquid amount observing device or the like. Furthermore, the tube pump, which is a consumable item having an unstable shape and therefore does not supply a stable flow amount, is controlled in a stable manner. Accordingly, this control can avoid a trouble of a decrease in the liquid to an amount smaller than a minimum liquid amount allowed to be handed by the device performing sorting or analysis of bioparticles even in a case of cleaning with a minimum liquid amount allowed to be handled within the closed channel.

In addition, the feedback control for maintaining a constant concentration of bioparticles on the basis of measurement values of the concentration measuring means 208 is adopted as described above. In this case, even when a plurality of tube pumps each supplying an unstable flow amount is used, the injection liquid amounts of the plurality of tube pumps can be synchronized with each other. Accordingly, the cleaning process can be executed with a small amount of liquid even in this case.

Note that PTL 1 presented above (U.S. Pat. No. 10,918,780) describes a technology associated with blood component donation which is a technology in a field different from the field of the present technology, and is not a technology including separating means using magnetic beads. However, this reference describes a method which measures liquid filtered by the hollow fiber membrane module 209, and controls the flow amount of the liquid on the basis of a result of comparison with a threshold. Meanwhile, according to the present embodiment, a concentration of a sample containing bioparticles is measured instead of a concentration of filtered liquid, and utilized for feedback for the tube pump. Accordingly, instability of the flow amount of the tube pump within the closed channel can be eliminated, and, therefore even cleaning of a small amount of liquid can be handled.

### (3) Automatic Process Effect Check

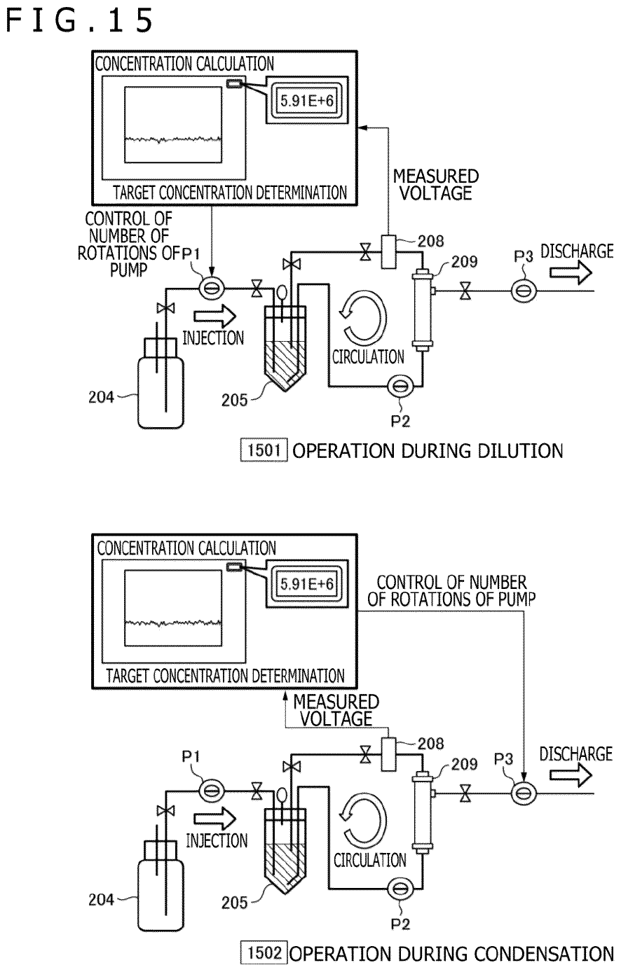

FIG. 15 is a diagram illustrating an example of an operation during concentration adjustment performed on an assumption that the hollow fiber membrane module 209 is used. In a case where a target concentration is set, concentration adjustment is generally achieved by dilution as indicated by 1501 in FIG. 15, and by condensation as indicated by 1502 in FIG. 15. However, if selection from completely different processes is incorrectly made on the basis of measurement values of a bioparticle concentration, which are close to a threshold and difficult to use for determination of the selection, an error of a non-arrival at a target concentration may be caused even by continuous execution of the concentration adjustment. For solving this drawback, a limit condensation process is introduced beforehand, and concentration adjustment of the dilution method is limited according to the present embodiment as described above in “(1) Stabilization of concentration adjustment flow.” Accordingly, advantageous effects of error prevention and time efficiency improvement of cleaning for bioparticles can be achieved.

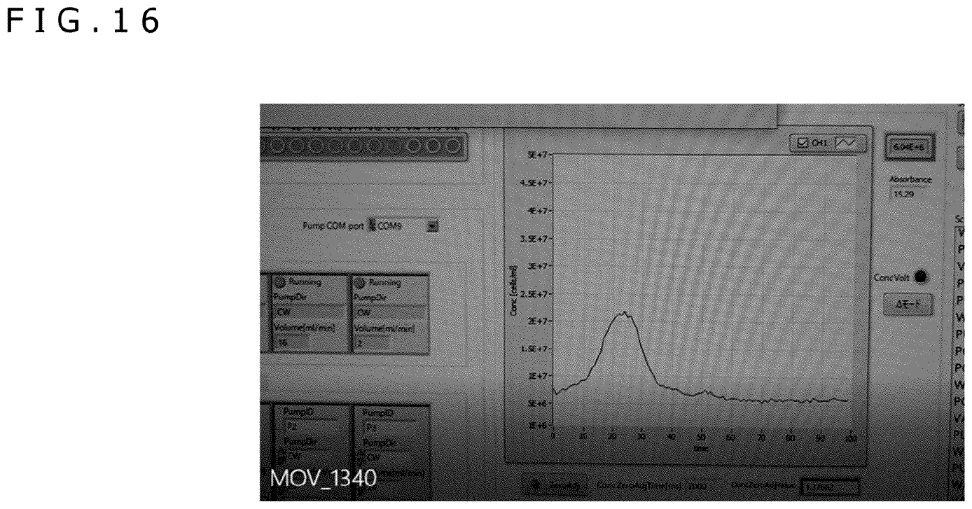

FIG. 16 is a photograph substituting for a drawing and illustrating an example of data of noise generated by dirt or bubbles in the closed channel during measurement by the concentration measuring means 208. As apparent from FIG. 16, a measurement value increases by generation of noise. Accordingly, during adjustment for increasing the concentration, an arrival at a target concentration may be incorrectly recognized due to this influence of dirt or bubbles. For solving this problem, bioparticle concentration adjustment is limited to dilution. For solving such a problem, adjustment limitation can also offer an advantageous effect of reduction of a process stop before an arrival at a target value caused by misrecognition of a measurement value as a result of noise.

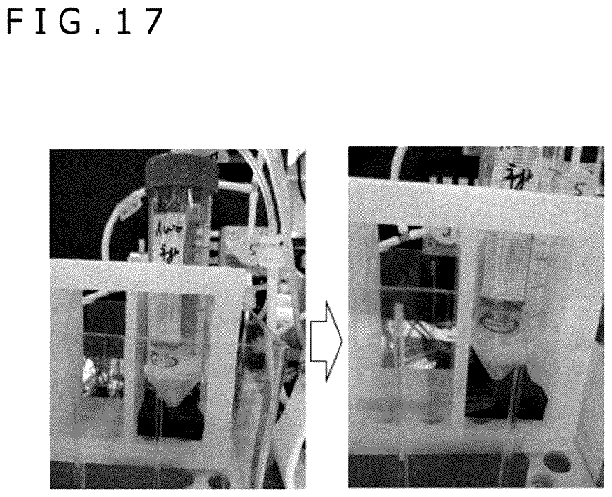

FIG. 17 is a photograph substituting for a drawing and illustrating an example of a liquid amount change produced when a cleaning process is executed for a sample containing bioparticles while setting the same number of rotations for the tube pump. An injection amount and a discharge amount of the tube pump are not equalized only by control of the number of rotations of the tube pump.

Accordingly, the liquid amount of the sample decreases within the reservoir 205. In this case, a concentration value corresponding to a measurement value of the concentration measuring means 208 increases. For solving this problem, feedback control is performed to maintain a constant level of the concentration, making it possible to avoid an increase or decrease of the liquid amount of the sample. Moreover, bubbles can be generated on a liquid surface of the sample containing bioparticles, and therefore a method of measuring a liquid amount on the basis of a height of the liquid surface is relatively unstable and is often unable to achieve accurate measurement. However, control based on the feedback control does not require measurement of the liquid amount on the basis of the height of the liquid surface, and thus can achieve stable measurement.

### 3. Second Embodiment (Closed Channel Type Bioparticle Processing Method)

A closed channel type bioparticle processing method according to the present technology (hereinafter also simply referred to as a “processing method according to the present technology”) performs a stirring step which stirs a sample, a separating step which uses magnetic beads each having a particle bonding substance, and a concentration measuring step which measures a concentration of bioparticles in the sample. The stirring step and the separating step are automatically switchable. The bioparticles are colored after separation of the bioparticles in the separating step. For example, the processing method according to the present technology may be performed by using the system 1 according to the present technology described in “1. First embodiment (closed channel type bioparticle processing system 1)” discussed above, or may be performed by a different system.

Moreover, the processing method according to the present technology may further perform a cleaning step which cleans the bioparticles and/or residues by using the hollow fiber membrane module 209. For example, the cleaning step may be performed by using the system 1 according to the present technology described in “1. First embodiment (closed channel type bioparticle processing system 1)” discussed above, or may be performed by a different system.

Furthermore, the processing method according to the present technology may adjust the concentration of the bioparticles in the concentration measuring step. For example, this step may be performed by using the system 1 according to the present technology described in “2. Automatic process using concentration measuring means” discussed above, or may be performed by a different system.

Note that the present technology may also be implemented in the following configurations.

[1]

A closed channel type bioparticle processing system including:
stirring means that stirs a sample;
separating means that uses magnetic beads each having a particle bonding substance; and
concentration measuring means that measures a concentration of bioparticles in the sample, in which
the stirring means and the separating means are automatically switchable, and
the bioparticles are colored after separation of the bioparticles by the separating means.
   [2]

The closed channel type bioparticle processing system according to [1], further including:
a hollow fiber membrane module.
   [3]

The closed channel type bioparticle processing system according to [2], in which the concentration of the bioparticles is adjusted by using the concentration measuring means.

[4]

The closed channel type bioparticle processing system according to [3], in which
a target concentration is set for the adjustment; and,
in a case where the measured concentration is higher than the target concentration, the bioparticles are diluted until the measured concentration reaches the target concentration or lower.
   [5]

The closed channel type bioparticle processing system according to [1], in which the concentration of the bioparticles within a closed channel is maintained at a constant concentration by using the concentration measuring means.

[6]

The closed channel type bioparticle processing system according to any one of [1] to [5], further including:
one or a plurality of pumps within a closed channel.
   [7]

The closed channel type bioparticle processing system according to [6], in which the pump is a tube pump.

[8]

The closed channel type bioparticle processing system according to [7], in which feedback control of an injection liquid amount of the tube pump is performed on the basis of a measurement value obtained by the concentration measuring means.

[9]

The closed channel type bioparticle processing system according to any one of [1] to [8], further including: three or more ports.

[10]

The closed channel type bioparticle processing system according to [2], in which a magnet is brought close to a channel located before entrance into the hollow fiber membrane module.

[11]

The closed channel type bioparticle processing system according to any one of [1] to [10], in which the particle bonding substances are antibodies or antibody fragments.

[12]

The closed channel type bioparticle processing system according to any one of [1] to [11], in which the bioparticles are cells.

[13]

The closed channel type bioparticle processing system according to any one of [1] to [12], in which the sample is a blood-derived sample.

[14]

The closed channel type bioparticle processing system according to any one of [1] to [13], in which the sample is configured not to communicate with an external environment.

[15]

A closed channel type bioparticle processing method including:
a stirring step that stirs a sample;
a separating step that uses magnetic beads each having a particle bonding substance; and
a concentration measuring step that measures a concentration of bioparticles in the sample, in which
the stirring step and the separating step are automatically switchable, and
the bioparticles are colored after separation of the bioparticles in the separating step.
   [16]

The closed channel type bioparticle processing method according to [15], further including:
a cleaning step that cleans the bioparticles and/or residues by using a hollow fiber membrane module.
   [17]

The closed channel type bioparticle processing method according to [15] or [16], in which the concentration of the bioparticles is adjusted in the concentration measuring step.

### Reference Signs List

1: Closed flow channel type bioparticle processing system
101: User interface
102: Recording unit
103: Liquid amount measuring means
104: Control unit
105, 208: Concentration measuring means
106, 206: Stirring means
107: Pump-valve
108, 207: Separating means
2: Closed channel type bioparticle processing device
200, 201, 202, 203, 204: Container
205: Reservoir
209: Hollow fiber membrane module
210: Different reservoir
211: Liquid waste container
212: Valve
213, P1, P2, P3: Pump
214: Separation magnet
215: Stage
216, 218, 220: Reagent container
217: Particle bonding substance container

1. A closed channel type bioparticle processing system comprising:
stirring means that stirs a sample;
separating means that uses magnetic beads each having a particle bonding substance; and
concentration measuring means that measures a concentration of bioparticles in the sample, wherein
the stirring means and the separating means are automatically switchable, and
the bioparticles are colored after separation of the bioparticles by the separating means.

2. The closed channel type bioparticle processing system according to claim 1, further comprising:
a hollow fiber membrane module.

3. The closed channel type bioparticle processing system according to claim 2, wherein the concentration of the bioparticles is adjusted by using the concentration measuring means.

4. The closed channel type bioparticle processing system according to claim 3, wherein
a target concentration is set for the adjustment; and,
in a case where the measured concentration is higher than the target concentration, the bioparticles are diluted until the measured concentration reaches the target concentration or lower.

5. The closed channel type bioparticle processing system according to claim 1, wherein the concentration of the bioparticles within a closed channel is maintained at a constant concentration by using the concentration measuring means.

6. The closed channel type bioparticle processing system according to claim 1, further comprising:
one or a plurality of pumps within a closed channel.

7. The closed channel type bioparticle processing system according to claim 6, wherein the pump is a tube pump.

8. The closed channel type bioparticle processing system according to claim 7, wherein an injection liquid amount of the tube pump is controlled on a basis of a measurement value obtained by the concentration measuring means.

9. The closed channel type bioparticle processing system according to claim 1, further comprising:
three or more ports.

10. The closed channel type bioparticle processing system according to claim 2, wherein a magnet is brought close to a channel located before entrance into the hollow fiber membrane module.

11. The closed channel type bioparticle processing system according to claim 1, wherein the particle bonding substances are antibodies or antibody fragments.

12. The closed channel type bioparticle processing system according to claim 1, wherein the bioparticles are cells.

13. The closed channel type bioparticle processing system according to claim 1, wherein the sample is a blood-derived sample.

14. The closed channel type bioparticle processing system according to claim 1, wherein the sample is configured not to communicate with an external environment.

15. A closed channel type bioparticle processing method comprising:
a stirring step that stirs a sample;
a separating step that uses magnetic beads each having a particle bonding substance; and
a concentration measuring step that measures a concentration of bioparticles in the sample, wherein
the stirring step and the separating step are automatically switchable, and
the bioparticles are colored after separation of the bioparticles in the separating step.

16. The closed channel type bioparticle processing method according to claim 15, further comprising:
a cleaning step that cleans the bioparticles and/or residues by using a hollow fiber membrane module.

17. The closed channel type bioparticle processing method according to claim 15, wherein the concentration of the bioparticles is adjusted in the concentration measuring step.
